(19) United States
(12) Patent Application Publication
(10) Pub. No.: US 2026/0120872 A1
(43) Pub. Date: Apr. 30, 2026

(54) THE METHOD AND APPARATUS FOR PREDICTING DISEASE OF COMPANION ANIMALS USING ARTIFICIAL NEURAL NETWORKS

(71) Applicant: FRESHOUR INC., Chungju-si (KR)

(72) Inventor: Kyeong Ho IM, Chungju-si (KR)

(73) Assignee: FRESHOUR INC., Chungju-si (KR)

(21) Appl. No.: 19/366,818

(22) Filed: Oct. 23, 2025

(30) Foreign Application Priority Data
Oct. 25, 2024 (KR) 10-2024-0147972

(51) Int. Cl. G16H 50/20; G06N 3/0455; G16H 50/70

(52) U.S. Cl. CPC G16H 50/20; G06N 3/0455; G16H 50/70

(57) ABSTRACT

There is disclosed a method for predicting a disease of a pet, comprising: acquiring animal health record information and microbiome information of pets; obtaining a Animal health record feature vector from the Animal health record information; obtaining a microbiome feature vector from the microbiome information; acquiring transformation information for converting the Animal health record feature vector into the microbiome feature vector; training a support vector machine using the microbiome feature vector and disease information; acquiring Animal health record information (A) input by a user; obtaining a Animal health record feature vector (A) with respect to the Animal health record information (A); converting the Animal health record feature vector (A) into a microbiome feature vector (A) using the transformation information; and predicting a disease by inputting the converted microbiome feature vector (A) into the trained support vector machine.

### CROSS-REFERENCE TO RELATED APPLICATION

This application claims priority to Korean Patent Application No. 10-2024-0147972 filed on Oct. 25, 2025, and all the benefits accruing therefrom under 35 U.S.C. § 119, the contents of which are incorporated by reference in their entirety.

### BACKGROUND

Embodiments disclosed herein relate generally to pet healthcare management and, more particularly, to methods and systems for predicting diseases of pets based on Animal health records (AHRs) and related biological information.

### SUMMARY

The pet industry has been rapidly growing as the number of households raising pets increases due to various factors such as the rise of single-person households, aging populations, and the pursuit of emotional stability. For example, not only traditional services such as pet grooming but also new and diverse services, including pet-related health insurance and pet healing services, are on the rise. In particular, as affection toward pets continues to grow, people's interest has shifted from grooming to health. Consequently, this growing concern is further extending to pet food.

However, pet food presents several challenges. Numerous manufacturers exist, and each manufacturer offers products with different nutritional compositions. Moreover, since pet food is not regulated as human food, the nutritional information provided on packaging is often inaccurate. In addition, it is difficult to accurately assess the condition of each pet. For these reasons, it remains challenging to determine and provide the most suitable type or amount of pet food tailored to the specific needs of a pet.

### BRIEF DESCRIPTION OF THE DRAWINGS

FIG. 1 is a diagram illustrating a method for predicting a disease of a pet based on Animal health record (AHR) information according to an embodiment.

FIG. 2 is a diagram illustrating AHR information of a pet according to an embodiment.

FIG. 3 is a diagram illustrating microbiome information of a pet according to an embodiment.

FIG. 4 is a flowchart illustrating a method for predicting a disease of a pet according to an embodiment.

FIG. 5 is a diagram illustrating a method for predicting a disease using AHR information and microbiome information according to an embodiment.

FIG. 6 is a diagram illustrating a method for obtaining feature vectors using an autoencoder according to an embodiment.

FIG. 7 is a diagram illustrating a method for training a support vector machine (SVM) according to an embodiment.

FIG. 8 is a diagram illustrating a method for predicting a disease using a trained SVM according to an embodiment.

FIGS. 9 and 10 are diagrams illustrating domain adaptation according to an embodiment.

FIG. 11 is a diagram illustrating an SVM according to an embodiment.

FIG. 12 is a diagram illustrating a method for training a second artificial neural network according to an embodiment.

FIG. 13 is a block diagram illustrating an example of a device for predicting a disease of a pet according to an embodiment.

With respect to the description of the drawings, the same or similar reference signs may be used for the same or similar elements.

### DETAILED DESCRIPTION OF EMBODIMENTS

The terminology used in the embodiments described herein has been selected to the extent possible from commonly used terms. However, such terminology may vary depending on the intent of those skilled in the art, judicial precedents, or the emergence of new technologies. In certain cases, terms arbitrarily selected by the applicant are also used, in which case the meanings of such terms will be described in detail in the relevant portions of the specification. Therefore, the terminology used in the specification should not be interpreted merely based on the literal name of the term but should be defined according to the meaning of the term and the overall content of the specification.

Throughout the specification, when a part is described as “comprising” a component, it should be understood, unless otherwise stated, that the part does not exclude other components but may further include additional components. Furthermore, ordinal terms such as “first” and “second” used in the specification are merely employed to distinguish between different components, and the components should not be limited by such terms. The terms may be used to distinguish one component from another.

Hereinafter, exemplary embodiments will be described in detail with reference to the accompanying drawings. However, it should be understood that the embodiments may be implemented in various forms and are not limited to the examples described herein.

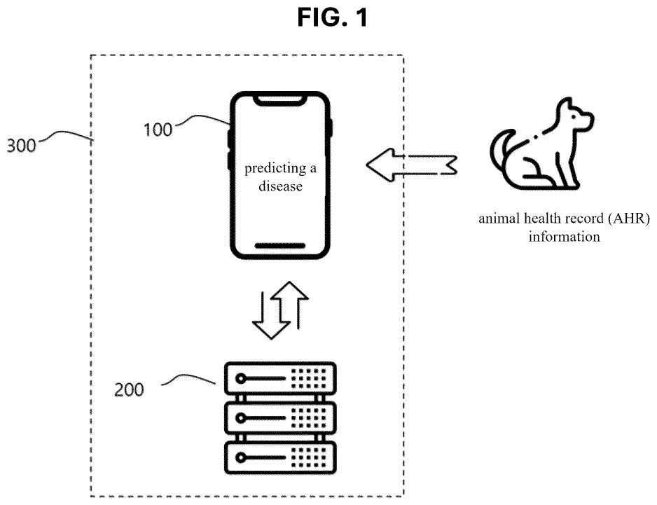

FIG. 1 is a diagram illustrating a method for predicting a disease of a pet based on AHR information according to an embodiment. Referring to FIG. 1, a device 300 may predict a disease of a pet based on AHR information. In other words, when a user inputs AHR results of a pet, the device 300 may predict a disease of the pet.

A mobile device 100 and/or a server 200 may predict a disease of a pet based on AHR information. A user may input AHR information of a pet through the mobile device 100. The mobile device 100 may transmit and receive data with the server 200. The device 300 may directly process the data to predict a disease, or alternatively, may transmit the data to the server 200 and receive the prediction result from the server 200. The server 200 may predict a disease based on the data received from the device 300.

Hereinafter, operations described as being performed by the device 300 may be interpreted as being performed solely by the mobile device 100 or jointly by the mobile device 100 and the server 200.

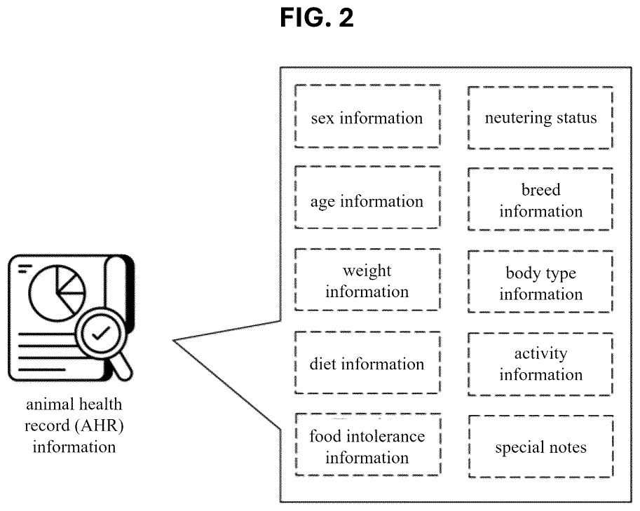

FIG. 2 is a diagram illustrating AHR information of a pet according to an embodiment. Referring to FIG. 2, the AHR information may include one or more of sex information, age information, weight information, diet information, food intolerance information, neutering status, breed information, body type information, activity information, and special notes. The device 300 may receive not only the AHR information illustrated in FIG. 2 but also various types of AHR information input from a user.

The sex information indicates whether the pet is male or female. The neutering status indicates whether the pet has been neutered. The age information indicates the age of the pet. A user may input the age of the pet or the date of birth through the device 300.

The breed information indicates the breed of the pet. A user may select one or more breeds of the pet. The device 300 may display categories such as dog, cat, etc., and receive an input of the pet type from the user. When the user selects a type, the device 300 may display breeds corresponding to the type. For example, if the user selects “dog,” the device 300 may display breeds such as Welsh Corgi, Border Collie, and Retriever.

The weight information indicates the weight of the pet. A user may input the weight of the pet through the device 300.

The body type information indicates the body shape of the pet. For example, the device 300 may display “very slim,” “slim,” “normal,” “overweight,” and “obese” and receive an input of the selected body type from the user.

The diet information indicates food or treats consumed by the pet. The diet information may include calorie information, nutritional data, and daily intake. The device 300 may receive an input of food types such as dry food, wet food, home-cooked food, raw food, and human food.

The activity information indicates the activity level or walking time of the pet. The device 300 may display “lethargic,” “low activity,” “normal,” and “high activity,” and receive an input of the selected activity level from the user. Additionally, the device 300 may receive an input of daily exercise time or walking time of the pet.

The food intolerance information indicates foods that the pet avoids or must not consume. The device 300 may display foods generally considered unsafe for pets and receive an input of the selected foods from the user. Additionally, the device 300 may receive inputs of food intolerance information via user-provided text or images.

The special notes information indicates diseases or habits of the pet. The device 300 may receive inputs of special notes through text or images provided by the user. For example, the special notes may include information on intestinal diseases, skin diseases, tear stains, or joint health.

In addition, the device 300 may receive health-related information. For example, the AHR information may further include whether the pet has experienced vomiting or diarrhea, whether the pet has taken probiotics, whether the pet has been administered medication, the type of medication administered, pregnancy status, allergy status, and types of allergies.

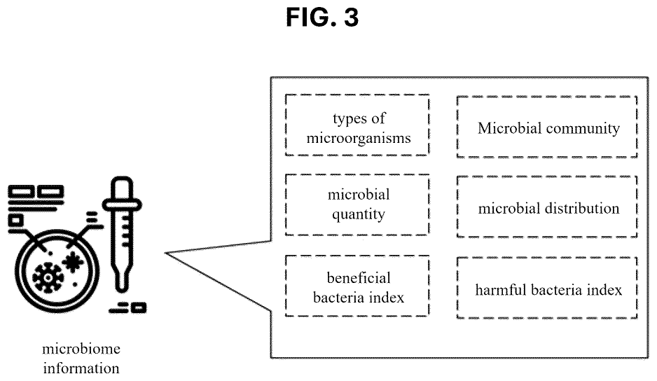

FIG. 3 is a diagram illustrating microbiome information of a pet according to an embodiment. Referring to FIG. 3, the microbiome information may include types of microorganisms, microbial community, microbial quantity, microbial distribution, beneficial bacteria index, and harmful bacteria index.

The microbiome information may represent information on intestinal microorganisms of a pet and may include bacteria, viruses, or other microorganisms detected from the pet. The microbiome information may be used to predict diseases of the pet.

The microbiome information may be acquired through a diagnostic device. For example, the diagnostic device may include diagnostic kits, microbiome-platform-based diagnostic devices, diagnostic devices for gastrointestinal diseases, pet healthcare diagnostic devices, fecal-based diagnostic devices, or fecal-based next generation sequencing (NGS) diagnostic devices.

Examples of microorganisms may include Lactobacillus, Clostridium perfringens, Blautia, Enterococcus faecium, Firmicutes, and Bacteroidetes. Beneficial bacteria may include Bifidobacterium, Lactococcus, Lactobacillus, and Streptococcus. Harmful bacteria may include Salmonella, Shigella, Clostridium difficile, Staphylococcus aureus, and Campylobacter jejuni.

The microbial quantity may represent an absolute or relative amount of microorganisms detected. The microbial distribution may represent the types and counts of microorganisms, or distribution patterns of microorganisms. The microbial community may represent types of microbial groups and the numbers of microorganisms included in each group. For example, when microorganisms are classified as beneficial, harmful, or neutral, the microbial community may represent the number of microorganisms included in each of the beneficial, harmful, and neutral groups. Since interacting microorganisms may vary depending on the pet, the microbial community may be set differently for each pet.

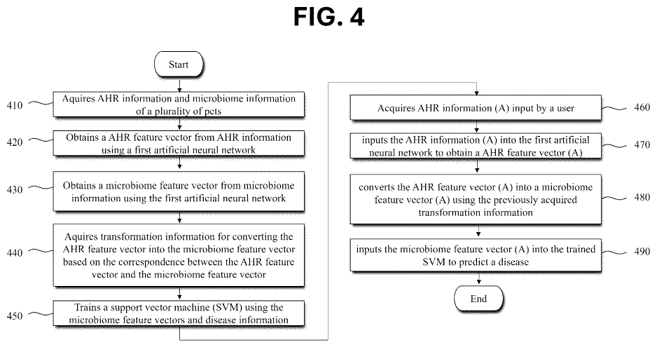

FIG. 4 is a flowchart illustrating a method for predicting a disease of a pet according to an embodiment. Referring to FIG. 4, the device 300 may predict diseases of a pet using an artificial neural network.

The device 300 performs preparation for disease prediction through steps 410 to 450. When the preparation is completed, the device 300 predicts a disease of AHR information (A) through steps 460 to 490. In other words, the device 300 may train an artificial neural network and an SVM using multiple AHR and microbiome data, obtain transformation information, and then predict a disease based on the user-provided AHR information (A).

In step 410, the device 300 acquires AHR information and microbiome information of a plurality of pets. The device 300 may store the AHR information and microbiome information in memory. The device 300 may also convert the AHR information and microbiome information into numerical data to be used as input or output data of the artificial neural network.

For example, the device 300 may acquire information of N pets, where N may be a natural number of 100 or more. The AHR information and the microbiome information acquired from the same pet correspond to each other. For instance, when first AHR information, first microbiome information, and first disease information are obtained from a first pet, the first AHR information, the first microbiome information, and the first disease information correspond to one another. The device 300 may acquire disease information of the pets, which may include obesity, diarrhea (non-hemorrhagic diarrhea, hemorrhagic diarrhea), constipation, and intestinal diseases. The device 300 may match microbiome information with disease information. Accordingly, when the device 300 acquires microbiome information of a pet, the device 300 may determine whether the pet has a disease.

In step 420, the device 300 obtains a AHR feature vector from AHR information using a first artificial neural network. The AHR feature vector represents a vector including only key information from the AHR information. For example, the first artificial neural network may be an autoencoder. The device 300 may input AHR information into the autoencoder to generate the AHR feature vector. The device 300 may generate N AHR feature vectors from N AHR datasets.

In step 430, the device 300 obtains a microbiome feature vector from microbiome information using the first artificial neural network. The microbiome feature vector represents a vector including only key information from the microbiome information. The device 300 may input microbiome information into the autoencoder to generate the microbiome feature vector. The device 300 may generate N microbiome feature vectors from N microbiome datasets.

When the device 300 generates the AHR feature vector using an autoencoder, the number of input nodes of the autoencoder may be determined according to the AHR information. Similarly, when the device 300 generates the microbiome feature vector using an autoencoder, the number of input nodes of the autoencoder may be determined according to the microbiome information. Therefore, the number of input nodes of the autoencoder may vary depending on the type of input data. However, the number of output nodes from which the feature vector is obtained may be the same regardless of the input type. In addition, the AHR feature vector and the microbiome feature vector may be vectors of the same dimension. In other words, the number of output nodes of the autoencoder may be fixed.

In step 440, the device 300 acquires transformation information for converting the AHR feature vector into the microbiome feature vector based on the correspondence between the AHR feature vector and the microbiome feature vector. The correspondence may represent a relationship between information acquired from the same pet. In other words, the AHR feature vector and the microbiome feature vector of the same pet correspond to each other. For example, when a first AHR feature vector corresponds to a first microbiome feature vector, the device 300 may obtain transformation information through a process of transforming the first AHR feature vector to match the first microbiome feature vector. In one example, the transformation information may be a vector. A vector that transforms the first AHR feature vector to match the first microbiome feature vector may serve as the transformation information.

In one embodiment, the device 300 may acquire the transformation information through domain adaptation. The device 300 may perform domain adaptation on a plurality of AHR feature vectors and a plurality of microbiome feature vectors. Through domain adaptation, the device 300 may transform the AHR feature vectors.

In another embodiment, the device 300 may acquire the transformation information using a second artificial neural network. In other words, the device 300 may train the second artificial neural network so that the network includes the transformation information. For example, the device 300 may input a first AHR feature vector as input data into the second artificial neural network and train the second artificial neural network so that a first microbiome feature vector is produced as output data. The device 300 may train the second artificial neural network using N AHR feature vectors and N microbiome feature vectors. The device 300 may then input a user-provided AHR feature vector (A) into the trained second artificial neural network to obtain a microbiome feature vector (A).

In step 450, the device 300 trains a support vector machine (SVM) using the microbiome feature vectors and disease information. The device 300 may classify the microbiome feature vectors based on disease information of the pets. That is, the device 300 may classify the microbiome feature vectors of pets having the same disease into the same group.

The device 300 may input microbiome feature vectors into the SVM and train the SVM so that disease information is output. For example, the device 300 may train the SVM using N microbiome feature vectors. The N microbiome feature vectors may be labeled with disease information. The SVM may be trained to classify microbiome feature vectors labeled with the same disease information into the same group. For example, if a first pet is obese, the device 300 may input a first microbiome feature vector into the SVM and train the SVM so that “obesity” is output. If a second pet has enteritis, the device 300 may input a second microbiome feature vector into the SVM and train the SVM so that “enteritis” is output.

In step 460, the device 300 acquires AHR information (A) input by a user. The user provides AHR information (A) of his or her pet (A). The AHR information (A) is not labeled with disease information. In other words, while the user does not know the disease of the pet (A), the device 300 predicts the disease of the pet (A) based solely on the AHR information (A).

In step 470, the device 300 inputs the AHR information (A) into the first artificial neural network to obtain a AHR feature vector (A). For example, the device 300 may input the AHR information (A) into an autoencoder to generate the AHR feature vector (A).

In step 480, the device 300 converts the AHR feature vector (A) into a microbiome feature vector (A) using the previously acquired transformation information. The transformation information is the information acquired in step 440 and may be the result of training through domain adaptation or the second artificial neural network. Since the AHR feature vector (A) has no corresponding microbiome feature vector, the device 300 generates the microbiome feature vector (A) using the transformation information.

In step 490, the device 300 inputs the microbiome feature vector (A) into the trained SVM to predict a disease. The trained SVM has been trained to classify N microbiome feature vectors according to disease information. Accordingly, the device 300 inputs the microbiome feature vector (A) into the SVM and confirms the output result, i.e., the predicted disease.

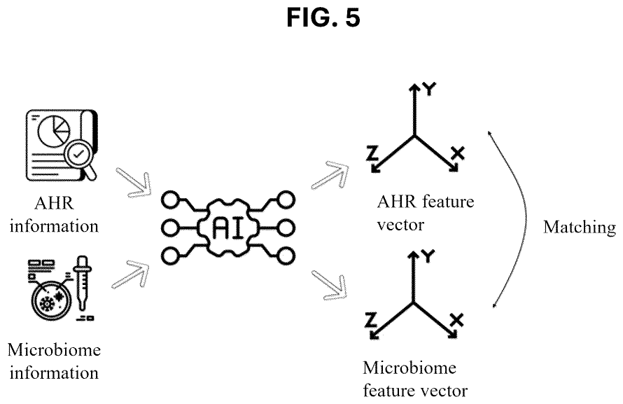

FIG. 5 is a diagram illustrating a method for predicting a disease using AHR information and microbiome information according to an embodiment. Referring to FIG. 5, the device 300 may input AHR information and microbiome information into the first artificial neural network to obtain respective feature vectors. AHR information and microbiome information of the same pet correspond to each other, and the AHR feature vector and the microbiome feature vector obtained through the neural network also correspond to each other. The corresponding information represents information of the same pet, and the corresponding vectors represent vectors obtained from the same pet.

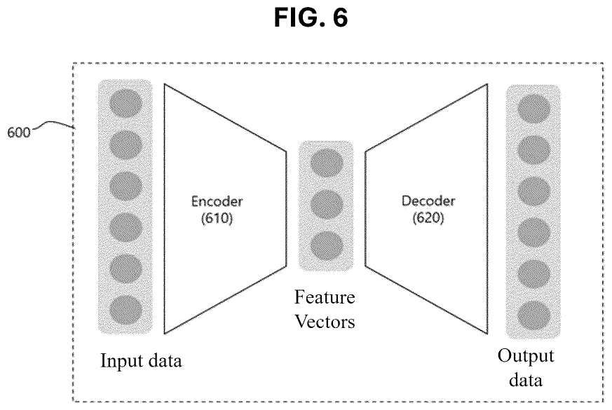

FIG. 6 is a diagram illustrating a method for obtaining feature vectors using an autoencoder according to an embodiment. The input data may be AHR information or microbiome information. An encoder 610 compresses the input data into a feature vector, and a decoder 620 restores the feature vector into output data. The autoencoder is trained so that the error between the input data and the output data is minimized.

In one example, the encoder 610 generates a feature vector having a smaller dimension than that of the input data. For example, when the dimension of the input data is K, the dimension of the feature vector may be L, where K is greater than L. When the input data is AHR information, the encoder 610 compresses the AHR information to generate a AHR feature vector. When the input data is microbiome information, the encoder 610 compresses the microbiome information to generate a microbiome feature vector.

In one example, the AHR feature vector and the microbiome feature vector may have the same dimension. In other words, the autoencoder generating the AHR feature vector and the autoencoder generating the microbiome feature vector may include the same number of nodes in the output layer that produces the feature vector.

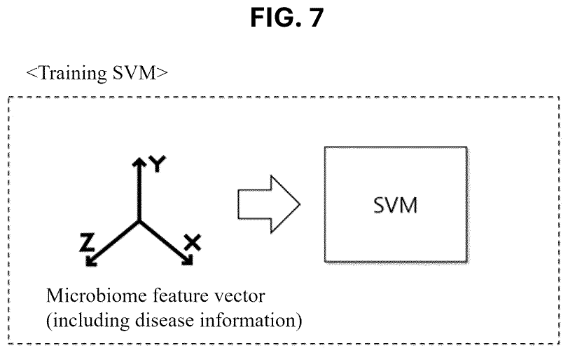

FIG. 7 is a diagram illustrating a method for training a support vector machine (SVM) according to an embodiment. The device 300 may train the SVM using a plurality of microbiome feature vectors. The microbiome feature vectors may be labeled with disease information. In other words, disease information of a pet may be matched to the microbiome feature vector of the pet. The device 300 may input the plurality of microbiome feature vectors into the SVM to classify the microbiome feature vectors. For example, the device 300 may determine a boundary that separates microbiome feature vectors of obese pets from microbiome feature vectors of pets with enteritis.

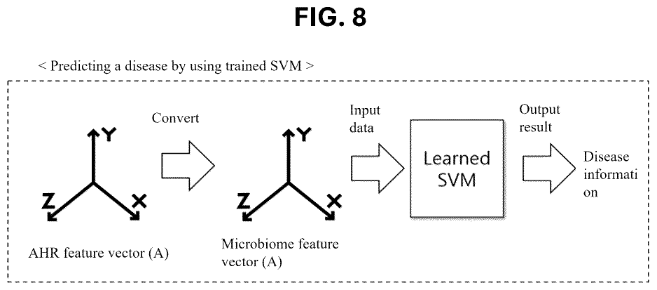

FIG. 8 is a diagram illustrating a method for predicting a disease using a trained SVM according to an embodiment. Referring to FIG. 8, the device 300 may predict a disease of a pet based solely on AHR information (A).

When the user inputs AHR information (A), the device 300 inputs the AHR information (A) into an autoencoder to generate a AHR feature vector (A). The device 300 then converts the AHR feature vector (A) into a microbiome feature vector (A) using the transformation information, or alternatively, inputs the AHR feature vector (A) into the trained second artificial neural network to obtain the microbiome feature vector (A). The second artificial neural network is trained using a plurality of AHR feature vectors and a plurality of microbiome feature vectors. The device 300 inputs a AHR feature vector into the second artificial neural network and trains the network so that the corresponding microbiome feature vector is output. That the two feature vectors correspond indicates that they are derived from the same pet.

The device 300 then inputs the microbiome feature vector (A) into the trained SVM to predict a disease. The SVM is trained using a plurality of microbiome feature vectors and corresponding disease information. The correspondence between a microbiome feature vector and disease information indicates that they are data of the same pet. Alternatively, it may be expressed that the microbiome feature vectors are labeled with disease information. The device 300 may train the SVM so that microbiome feature vectors labeled with the same disease information are classified into the same group.

The trained SVM outputs disease information when a microbiome feature vector (A) is input. The trained SVM determines to which group the input microbiome feature vector (A) belongs and outputs disease information corresponding to the determined group.

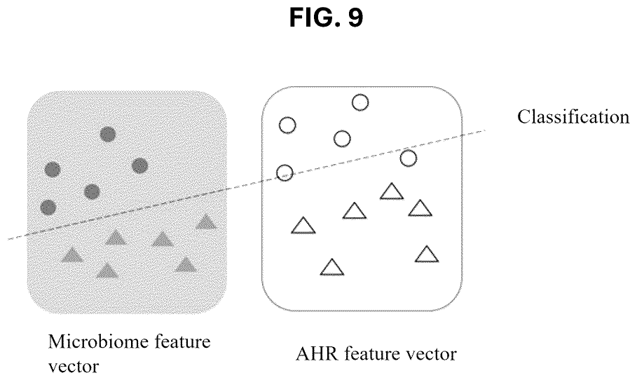

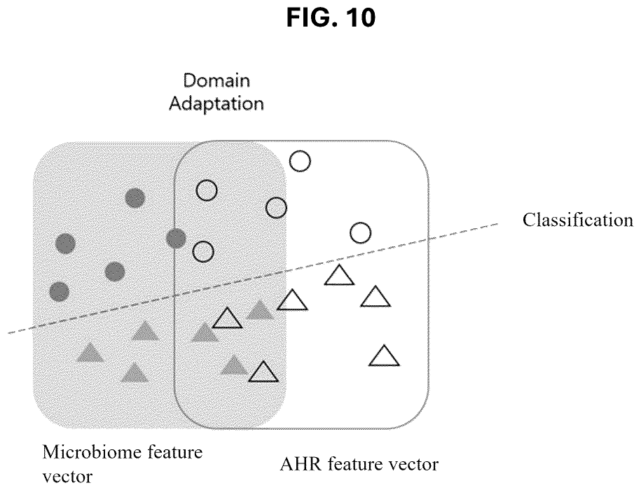

FIGS. 9 and 10 are diagrams illustrating domain adaptation according to an embodiment. The device 300 may transform AHR feature vectors through domain adaptation.

For example, feature vectors indicated by circles may be labeled with a first disease, and feature vectors indicated by triangles may be labeled with a second disease. In FIG. 9, the dotted line represents a decision boundary for classifying microbiome feature vectors. The dotted line classifies microbiome feature vectors into two groups. However, when the same dotted line is applied to AHR feature vectors, the AHR feature vectors are not accurately classified.

FIG. 10 illustrates a case where the AHR feature vectors are transformed. When the AHR feature vectors are transformed through domain adaptation, both microbiome feature vectors and AHR feature vectors may be classified by the same decision boundary.

The device 300 may store, in memory, transformation information for transforming AHR feature vectors so that microbiome feature vectors and AHR feature vectors are classified according to the same criterion. When a AHR feature vector (A) is input, the device 300 may transform the AHR feature vector (A) using the stored transformation information.

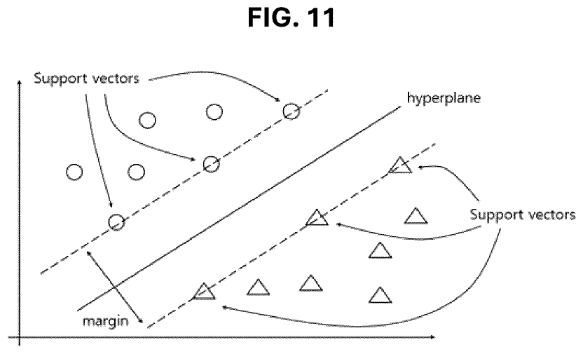

FIG. 11 is a diagram illustrating an SVM according to an embodiment. Referring to FIG. 11, the device 300 may use a plurality of microbiome feature vectors as training data for the SVM to obtain classification criteria according to diseases. Each microbiome feature vector is labeled with its corresponding disease.

A support vector refers to a vector located at the outermost boundary among vectors belonging to different categories. The device 300 may calculate a margin based on the support vectors. The margin represents a distance between categories determined using the support vectors. A hyperplane represents a plane that maximizes the margin between the support vectors.

For example, circles may represent microbiome feature vectors of pets having a first disease, and triangles may represent microbiome feature vectors of pets having a second disease. Accordingly, the device 300 may obtain a hyperplane separating the circles and triangles using the SVM.

Although FIG. 11 illustrates an example with only two diseases, pets may have three or more diseases, and the device 300 may classify microbiome feature vectors into three or more disease categories using the SVM.

The device 300 trains the SVM using microbiome feature vectors of pets whose diseases are already known. The device 300 may train the SVM using a plurality of microbiome feature vectors. The device 300 may then use the trained SVM to predict a disease of a pet whose disease is not identified. The device 300 inputs a microbiome feature vector (A) of such a pet into the SVM and acquires disease information classified by the SVM. Accordingly, the device 300 predicts the disease of the pet based on the classification result of the SVM. In this case, the microbiome feature vector (A) may be obtained by transforming a AHR feature vector (A).

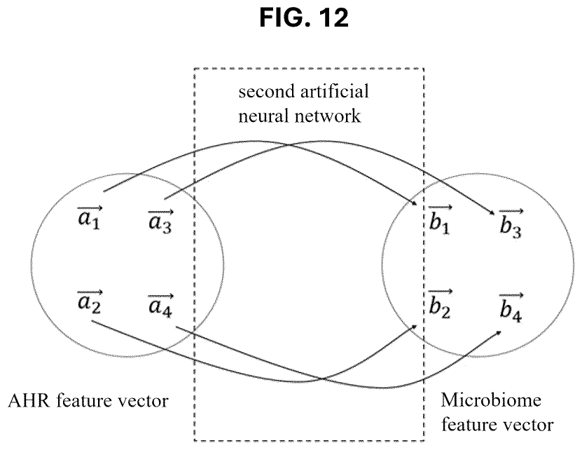

FIG. 12 is a diagram illustrating a method for training a second artificial neural network according to an embodiment. Referring to FIG. 12, the device 300 may generate the second artificial neural network that matches AHR feature vectors to microbiome feature vectors. In other words, the device 300 may generate a trained second artificial neural network.

The device 300 may train the second artificial neural network using a plurality of AHR feature vectors and microbiome feature vectors. The device 300 may perform supervised learning by inputting AHR feature vectors into the second artificial neural network as input data and generating microbiome feature vectors as output data. The AHR feature vectors and microbiome feature vectors may be training data, and they are matched with each other. Accordingly, when the device 300 inputs a first AHR feature vector into the second artificial neural network, the network may output a first microbiome feature vector. Similarly, when the device 300 inputs a second AHR feature vector into the second artificial neural network, the network may output a second microbiome feature vector. Although FIG. 12 illustrates only four vectors for ease of explanation, the number of feature vectors for training may be in the thousands or tens of thousands.

In one example, the second artificial neural network may be a multilayer perceptron (MLP). The second artificial neural network may include an input layer, hidden layers, and an output layer, and may include two or more hidden layers.

The trained second artificial neural network may be used to generate a microbiome feature vector (A). The device 300 may receive AHR information (A) from a user, input the AHR information (A) into an autoencoder to obtain a AHR feature vector (A), and then input the AHR feature vector (A) into the trained MLP to obtain a microbiome feature vector (A). The device 300 may then input the microbiome feature vector (A) into the trained SVM to obtain disease information.

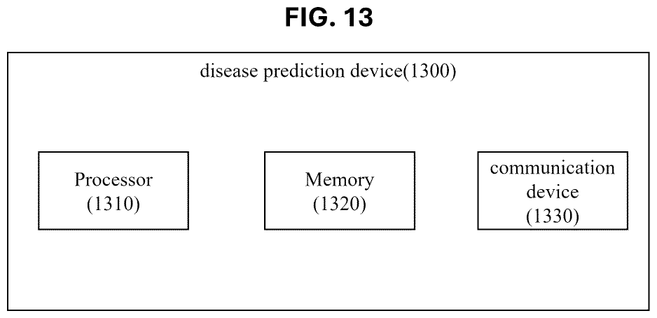

FIG. 13 is a block diagram illustrating an example of a device for predicting a disease according to an embodiment. Referring to FIG. 13, a disease prediction device 1300 includes a processor 1310, a memory 1320, and a communication device 1330. The operations described in FIGS. 1 to 12 may be performed by the disease prediction device 1300.

The processor 1310 may process instructions of a computer program by performing basic arithmetic, logic, and input/output operations. The instructions may be provided from the memory 1320 or from an external device.

For example, functions performed by respective modules included in the processor 1310 may be executed by a single processor or may be distributed among separate processors. The processor 1310 may execute operations or data processing related to control and/or communication of at least one other component.

The processor 1310 may be implemented as an array of multiple logic gates, or as a combination of a general-purpose microprocessor and a memory storing a program executable by the microprocessor. For example, the processor 1310 may include a general-purpose processor, a central processing unit (CPU), a graphics processing unit (GPU), a neural processing unit (NPU), a microprocessor, a digital signal processor (DSP), a controller, a microcontroller, or a state machine. In certain environments, the processor 1310 may further include an application-specific integrated circuit (ASIC), a programmable logic device (PLD), or a field-programmable gate array (FPGA). For instance, the processor 1310 may refer to a combination of processing devices such as a digital signal processor (DSP) combined with a microprocessor, a combination of multiple microprocessors, one or more microprocessors combined with a DSP core, or any other such configurations.

The memory 1320 may include any non-transitory computer-readable recording medium. In one example, the memory 1320 may include permanent mass storage devices such as random access memory (RAM), read-only memory (ROM), disk drives, solid state drives (SSDs), or flash memory. In another example, non-volatile mass storage devices such as ROM, SSDs, flash memory, and disk drives may be separate permanent storage devices distinct from the memory. The memory 1320 may further store an operating system (OS) and at least one program code (e.g., code that enables the processor 1310 to perform operations described below with reference to the drawings).

Such software components may be loaded into the memory 1320 from a computer-readable recording medium separate from the memory. The separate computer-readable recording medium may be directly connected to the computer, and may include, for example, a floppy drive, disk, tape, DVD/CD-ROM drive, or memory card. Alternatively, the software components may be loaded into the memory 1320 through the communication device 1330 rather than from a computer-readable recording medium. For example, at least one program may be loaded into the memory 1320 based on installation files of an application provided via the communication device 1330 by developers or a file distribution system distributing application installation files.

The communication device 1330 performs data communication between the vehicle 10 and an external device. For example, the communication device 1330 may communicate with the controller 40, the server 30, and/or the station 50 using various communication methods such as infrared communication, radio frequency (RF) communication, Wi-Fi communication, ZigBee communication, Bluetooth communication, laser communication, ultra-wideband (UWB) communication, LTE, 5G, 6G, or wireless LAN. However, the communication method employed by the communication device 1330 is not limited to those described above.

Meanwhile, the above-described methods may be embodied as programs executable by a computer and may be implemented in a general-purpose digital computer that operates such programs using a computer-readable recording medium. In addition, data structures used in the above-described methods may be recorded on the computer-readable recording medium by various means. The computer-readable recording medium may include magnetic storage media (e.g., ROM, RAM, USB, floppy disk, hard disk), and optical storage media (e.g., CD-ROM, DVD), among others.

It will be understood by those skilled in the art that various modifications may be made without departing from the essential characteristics of the disclosure. Therefore, the methods disclosed herein should be considered illustrative rather than restrictive, and the scope of protection is defined by the appended claims rather than the foregoing description, with all equivalents thereof being encompassed within the scope of the claims.

According to various embodiments, each component (e.g., a module or a program) of the above-described components may include a single subject or multiple subjects. According to various embodiments, one or more components or operations of the above-described components may be omitted, or one or more other components or operations may be added. Alternatively or additionally, a plurality of components (e.g., modules or programs) may be integrated into a single component. In such a case, the integrated component may still perform one or more functions of each of the plurality of components in the same or similar manner as those performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A method for predicting a disease of a pet, comprising:
acquiring, as training data, Animal health record information and microbiome information of a plurality of pets;
obtaining, by a first artificial neural network, a Animal health record feature vector from the Animal health record information;
obtaining, by the first artificial neural network, a microbiome feature vector from the microbiome information;
acquiring transformation information for converting the Animal health record feature vector into the microbiome feature vector, based on a correspondence between the Animal health record feature vector and the microbiome feature vector;
training a support vector machine using the microbiome feature vector and disease information;
acquiring, as inference data, Animal health record information (A) input by a user;
obtaining, by the first artificial neural network, a Animal health record feature vector (A) with respect to the Animal health record information (A);
converting the Animal health record feature vector (A) into a microbiome feature vector (A) using the transformation information; and
predicting a disease by inputting the converted microbiome feature vector (A) into the trained support vector machine.

2. The method of claim 1, wherein the first artificial neural network is an autoencoder.

3. The method of claim 1, wherein the microbiome information included in the training data is labeled with disease information of the pets, and training the SVM comprises classifying the microbiome feature vectors according to the disease information.

4. The method of claim 1, wherein the transformation information comprises information for converting the AHR feature vector into the microbiome feature vector, and the correspondence comprises the AHR feature vector and the microbiome feature vector of the same pet.

5. The method of claim 1, wherein obtaining the transformation information comprises training a second artificial neural network by inputting the AHR feature vector into the second artificial neural network such that the microbiome feature vector is output from the second artificial neural network.

6. The method of claim 5, wherein the second artificial neural network is a multilayer perceptron (MLP).

7. A non-transitory computer-readable medium storing instructions that, when executed by a computer, cause the computer to perform the method of claim 1.

8. An apparatus for predicting a disease of a pet, comprising: and
at least one processor coupled to the memory and configured to execute instructions stored in the memory to:
acquire, as training data, AHR information and microbiome information of a plurality of pets;
generate, by a first artificial neural network, a AHR feature vector and a microbiome feature vector from the training data;
obtain transformation information based on a correspondence between the AHR feature vector and the microbiome feature vector of the same pet;
train a support vector machine (SVM) using the microbiome feature vectors and disease information;
acquire, as inference data, AHR information (A) input by a user;
generate, by the first artificial neural network, a AHR feature vector (A) from the user-provided AHR information (A);
convert the AHR feature vector (A) into a microbiome feature vector (A) using the transformation information; and
predict a disease by inputting the microbiome feature vector (A) into the trained SVM.

9. The apparatus of claim 8, wherein the first artificial neural network is an autoencoder.

10. The apparatus of claim 8, wherein the microbiome information included in the training data is labeled with disease information of the pets, and the processor is configured to train the SVM to classify the microbiome feature vectors according to the disease information.

11. The apparatus of claim 8, wherein the transformation information comprises information for converting the AHR feature vector into the microbiome feature vector, and the correspondence comprises the AHR feature vector and the microbiome feature vector of the same pet.

12. The apparatus of claim 8, wherein the processor is configured to train a second artificial neural network such that, when the AHR feature vector is input, the microbiome feature vector is output.

13. The apparatus of claim 12, wherein the second artificial neural network is a multilayer perceptron (MLP).
